(19) United States
(12) Patent Application Publication
(10) Pub. No.: US 2004/0122280 A1
(43) Pub. Date: Jun. 24, 2004

(54) Optical probes

(76) Inventor: Robert W. Forney, Waltham, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/325,515

(22) Filed: Dec. 19, 2002

(51) Int. Cl. A61N 5/02

(52) U.S. Cl. 600/2

(57) ABSTRACT

Optical probes that may withstand a variety of hostile environments and that may provide high optical throughput are described herein. According to one exemplary embodiment, an optical probe may include a tube, an optical element, a cap, and a spring. The tube may have a first end, a second end, and an inner surface. The optical element may be insertable into the tube through the first end and may have an outer surface. A portion of the tube inner surface and a portion of the optical element outer surface may be matably tapered to provide a substantially fluid tight seal between the tube and the optical element. The cap may be attachable to the first end. The spring may be compressible between the optical element and the cap.

### FIELD

The present disclosure relates to devices for detecting electromagnetic radiation.

### BACKGROUND

An optical probe is a device that may be used to detect electromagnetic radiation emitted by an area of interest.

A variety of conventional optical probes are presently available. Conventional optical probes may be used to detect electromagnetic radiation emitted by substances flowing in closed pathways, such as closed pathways found in chemical production feed lines, polymer extruders, reactors, and other industrial processes. Chemical production feed lines, polymer extruders, reactors, and other industrial processes may be characterized by hostile environments that can include, for example, temperature cycling, pressure cycling, mechanical shock or vibration, and/or other external forces, such as corrosion and viscous fluid flow. Many conventional optical probes cannot withstand such hostile environments, thereby inhibiting their utility.

### SUMMARY

Optical probes that may withstand a variety of hostile environments and that may provide enhanced optical throughput are described herein.

According to one exemplary embodiment, an optical probe may include a tube, an optical element, a cap, and a spring. The tube may have a first end, a second end, and an inner surface. At least a portion of the tube inner surface may be tapered. The optical element may be insertable into the tube through the first end and may have an outer surface. At least a portion of the optical element outer surface may be tapered. The tapered portion of the optical element outer surface may be mated to the tapered portion of the tube inner surface to provide a substantially fluid tight seal between the tube and the optical element. The cap may be attachable to the first end. The spring may be compressible between the optical element and the cap.

In one aspect of the exemplary embodiment, the second end may include an aperture permitting transmission of electromagnetic radiation out of the tube.

In another aspect of the exemplary embodiment, at least a portion of the tube inner surface may be stepped, and the spring may be removeably and replaceably insertable into the stepped portion of the tube inner surface. The stepped portion of the tube inner surface may be disposed adjacent to the tapered portion of the tube inner surface. The stepped portion of the tube inner surface may be stepped outward from the tube inner surface.

In another aspect of the exemplary embodiment, the tapered portion of the tube inner surface may include a taper extending outward from the tube inner surface.

In another aspect of the exemplary embodiment, the tapered portion of the tube inner surface may include a taper that spans a planar angle less than approximately 10 degrees. The taper may span a planar angle of approximately 3 degrees.

In another aspect of the exemplary embodiment, at least one of the tapered inner surface of the tube and the tapered outer surface of the optical element may include at least one of a gasket and a layer of at least one of gold, graphite, platinum, and polytetrafluoroethylene (PTFE).

In another aspect of the exemplary embodiment, the optical element may include at least one of a window for transmitting electromagnetic radiation and a lens for focusing electromagnetic radiation. The window and the lens may be integrally formed. The window and the lens may be attached to each other using at least one of an adhesive and a fastener.

In another aspect of the exemplary embodiment, the spring may include at least one of a spring washer, a C-ring, and a metal-loaded gasket.

In another aspect of the exemplary embodiment, the cap may be attached to the first end using at least one of an adhesive, a braze, a fastener, a thread, and a weld. The cap may-be welded to the first end under a load of at least approximately 150 psi.

In another aspect of the exemplary embodiment, the cap may include a washer.

In another aspect of the exemplary embodiment, the tube may include at least one of a ceramic, a polymer, and a metal.

In another aspect of the exemplary embodiment, the optical element may include at least one of diamond, germanium, glass, plastic, potassium bromide, quartz, sapphire, silicon, sodium chloride, zinc selenide, and zinc sulfide.

### BRIEF DESCRIPTION OF THE DRAWINGS

These and other features and objects of the invention will be more fully understood from the following detailed description that should be read in light of the accompanying drawings. In the accompanying drawings, like reference numerals refer to like parts throughout the different views. While the drawings illustrate principles of the invention disclosed herein, they are not drawn to scale, but show only relative dimensions.

FIG. 1 is an exploded longitudinal cross-sectional view of an exemplary embodiment of an optical probe described herein.

FIG. 2 is a longitudinal cross-sectional view of an exemplary system for Raman spectroscopy, including an exemplary embodiment of an optical probe described herein.

### DETAILED DESCRIPTION

Certain exemplary embodiments will now be described to provide an overall understanding of the optical probes described herein. One or more examples of the exemplary embodiments are shown in the drawings. Those of ordinary skill in the art will understand that the optical probes described herein can be adapted and modified to provide devices, methods, schemes, and systems for other applications, and that other additions and modifications can be made to the optical probes described herein without departing from the scope of the present disclosure. For example, components, features, modules, and/or aspects of the exemplary embodiments can be combined, separated, interchanged, and/or rearranged to generate other embodiments. Such modifications and variations are intended to be included within the scope of the present disclosure.

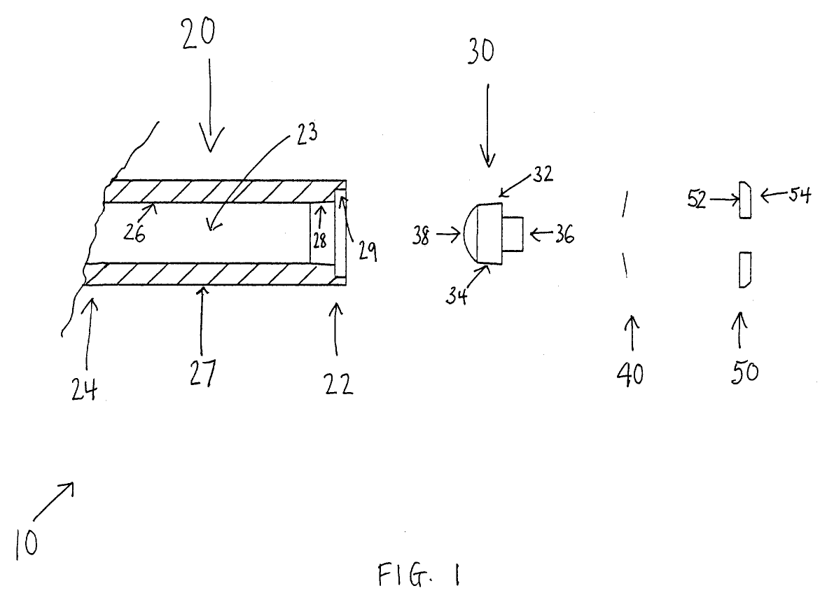

An exemplary embodiment of an optical probe described herein is shown in FIG. 1. As shown in FIG. 1, an optical probe 10 may include a tube 20, an optical element 30 for detecting and/or focusing and/or transmitting electromagnetic radiation emitted by an area of interest, a cap 50 for inhibiting the entrance of fluids into the tube 20, and a spring 40 compressible between the optical element 30 and the cap 50. As shown, the tube 20 may have a first end 22, a second end 24, an outer surface 27, an inner surface 26, and the inner surface 26 may include a tapered-portion 28. As shown, in one embodiment, the tube 20 may have a stepped portion 29 located next to the tapered portion 28. In the exemplary embodiment, as shown in FIG. 1, the optical element 30 may be insertable into the tube 20 through the first end 22 and may have an outer surface 32, and the outer surface 32 may include a tapered portion 34. As shown, the cap 50 may be attachable to the first end 22 of the tube 20, and the spring 40 may be compressible between the optical element 30 and the cap 50. As shown, in one embodiment, the spring 40 may be removeably and replaceably insertable into the stepped portion 29 of the tube 20. In the shown embodiment, the tapered portions 28, 34 of the tube inner surface 26 and the optical element outer surface 32 may be matably tapered, so that a substantially fluid tight seal may be formed between the tube 20 and the optical element 30. Potentially advantageously, as shown in FIG. 1, the cap 50 may provide pressure on the spring 40, and the spring 40 may provide pressure on the optical element 30 in a direction to strengthen the substantially fluid tight seal, thereby providing an optical probe 10 including a tube-to-optical element seal that may withstand a variety of hostile environments.

As shown in FIG. 1, the tapered portion 28 of the tube inner surface 26 may include a taper extending in a direction substantially outward from the tube inner surface 26, and the tapered portion 34 of the optical element outer surface 32 may include a mated taper extending in a direction substantially outward from the optical element outer surface 32. A variety of mated tapers are available for the tapered portions 28, 34. In the shown embodiment, the taper of the tapered portion 28 may span a planar angle of approximately 3 degrees, measured with respect to the tube inner surface 26. In other embodiments, the taper may span a planar angle less than approximately 90 degrees, and preferably less than 10 degrees.

In one embodiment, the tapered portion 28 of the tube inner surface 26 may extend in a direction substantially inward from the tube inner surface 26, and the tapered portion 34 of the optical element outer surface 32 may include a mated taper, extending in a direction substantially inward from the optical element outer surface 32. In such an embodiment, the optical element 30 may be inserted into the tube 20 through the second end 24, the cap 50 may be attached at the second end 24, and the spring 40 may be disposed between cap 50 and optical element 30.

In one embodiment, at least one of the tapered portions 28 and 34 may include a coating of material to act as a gasket and thereby enhance the substantially fluid tight seal between the tube 10 and the optical element 30. The coating may also enhance the chemical and thermal resistance of the substantially fluid tight seal to hostile environments. The coating may include a layer of at least one of gold, graphite, platinum, PTFE, other corrosion-resistant materials, and a combination of the foregoing. The coating may be applied to the tapered portion 28 and/or the tapered portion 34 by using well known schemes, for example, electroplating, sputtering, and/or vacuum deposition.

In one embodiment, at least one of the tapered portions 28 and 34 may include a gasket. The gasket may be constructed from a polymer, such as an elastomer. The gasket may include an o-ring.

As shown in FIG. 1, in one embodiment, the tube inner surface 26 may include a stepped portion 29 for removeably and replaceably receiving the spring 40. In the shown embodiment, the stepped portion 29 may be stepped outward from the tube inner surface 26. Generally, the tube inner surface 26 may include a variety of otherwise shaped portions for receiving the spring 40. For example, in place of the stepped portion 29, the tube inner surface 26 may include another tapered portion tapered outward from the tube inner surface 26, or may include an arcuate portion curved outward from the tube inner surface 26, or may include a multiply stepped portion including multiple steps outward from the tube inner surface 26, or a combination of the foregoing.

As shown in FIG. 1, the tapered portion 28 and the stepped portion 29 of the tube inner surface 26 may be positioned adjacent to each other. In one embodiment, the tapered portion 28 and the stepped portion 29 may be separated from each other to accommodate optical elements 30 and/or springs 40 having a variety of shapes and sizes. Additionally, the tapered portion 28 and-the stepped portion 29 may have a variety of relative widths to accommodate different optical elements 30 and/or springs 40.

A variety of constructions are available for the tube 20. Generally, the tube may be constructed from at least one of a ceramic, a polymer, and a metal. Metals may include pure metals and alloys. Alloys may include alloys based on at least one of chromium, iron, molybdenum, and nickel. Alloys may include stainless steel, Hastelloy™, Iconel™. Alloys may also include chromium, nickel-chromium, nickel-molybdenum, nickel-chromium-molybdenum, and iron-chromium-nickel alloys. Generally, the tube 20 may be constructed from one or more materials suitable for constructing an optical probe. As shown in FIG. 1, the tube 20 may have a rectangular longitudinal cross-section. Alternately, the tube 20 may have a variety of longitudinal and transverse cross-sections, and may have a variety of shapes when viewed from above. For example, the tube may have a substantially polygonal, oval, or semi-oval cross-section, and a substantially polygonal, oval, or semi-oval shape when viewed from above.

As shown in FIG. 1, the spring 40 may be compressible between the optical element 30 and the cap 50. As shown in FIG. 1, the spring 40 may be oriented to exert a pressure on the optical element 30 in a direction to strengthen the substantially fluid tight seal between the optical element 30 and the tube 20. In the shown embodiment, the cap 50 may be attached to the first end 22 of the tube 20 under a load that compresses the spring 40, and the spring 40 may tend to push the optical element 30 towards the interior 23 of the tube 20. Potentially advantageously, the optical probe 10, including the tube 20, optical element 30, spring 40, and cap 50 arranged as shown, may provide a substantially fluid tight seal that may withstand a variety of hostile environments.

A variety of springs may be used with the embodiment shown in FIG. 1. For example, the spring 40 may include a spring washer, such as a Belleville spring washer, a curved spring washer, a finger spring washer, or a wave spring washer. Also, the spring 40 may include a C-ring or a metal-loaded gasket. In the shown embodiment, the spring 40 may be a Belleville spring washer having an orientation as indicated in the drawing. In various embodiments, one or more springs 40 may be used to provide pressure on the optical element 30, and the one or more springs may be removeably and replaceably disposed in the stepped portion 29.

As shown in FIG. 1, the cap 50 may be attached to the first end 22 of the tube 20 to provide pressure on the spring 40 and inhibit the entrance of fluids into the tube 20 through the first end 22. A variety of schemes may be used to attach the cap 50 to the first end 22 of the tube 20. For example, the cap 50 may be attached by using an adhesive, a braze, a fastener, a thread, a weld, or another suitable scheme. The cap 50 may be removeably and replaceably attached,to the first end 22 to facilitate replacement of the optical element 30 and/or the spring 40. In one embodiment, the second end 22 of the tube 20 and the cap 50 may include complementary threads, and the cap 50 may be screwed onto the second end 22. For example, the outer surface 27 of the tube may include threads, and the cap 50 may include a sidewall extending from a base, in which the sidewall may include an inner surface with complementary threads. In such an embodiment, the cap 50 may be screwed onto the second end 22 of the tube 20 so that the sidewall of the cap 50 surrounds at least a portion of the outer surface 27 of the tube 20. In one embodiment, the cap 50 may be welded to the first end 22 of the tube 24 under a load of at least approximately 150 psi and, preferably, under a load between approximately 150 psi and approximately 500 psi. In such an embodiment, the tube 20 may be held in a clamp or other compression-type mechanism that can compress the cap 50 onto the washer 40. The cap 50 may be tack welded onto the tube 20. The clamp may be removed, and the cap 50 may be further welded onto the tube 20.

As shown in FIG. 1, the cap 50 may be a beveled washer having a flat surface 52 and a beveled surface 54. A variety of other caps 50 are also available. For example, the cap 50 may be a washer having flat and/or beveled surfaces or another suitable device for providing pressure on the spring 40 and inhibiting the entrance of fluids into the tube 20 through the first end 22. As previously indicated, the cap 50 may include threads for threadably mounting the cap 50 onto the tube 20. In such an embodiment, the cap 50 may have a base and a sidewall extending upward from the base. The sidewall may include an inner surface, and the inner surface may have threads that are complementary to threads on an outer surface 27 of tube 20.

As shown in FIG. 1, the optical probe 10 may include an optical element 30 for detecting and/or focusing and/or transmitting electromagnetic radiation. More specifically, the optical probe 10 and the optical element 30 may be used to assist an observer, such as a human observer or a machine, including a machine capable of being controlled by a processor, to detect, focus, measure, observe, see, or otherwise transmit or view electromagnetic radiation emitted by an area of interest. In the shown embodiment, the optical element 30 may include a flat face 36 facing away from the interior 23 of the tube 20 and a curved face 38 facing towards the interior 23 of the tube 20. In the shown embodiment, the flat face 36 may comprise a window for detecting and/or observing and/or transmitting the electromagnetic radiation emitted from the area of interest, and the curved face 38 may comprise a lens for focusing the electromagnetic radiation emitted by the area of interest towards the interior 23 of the tube 20.

A variety of other optical elements 30 are available for the optical probe 10. In various embodiments, the optical element 30 may include one or more windows and/or one or more lenses. In one embodiment, the optical element 30 may include two lenses, in which one lens faces the interior 23 of the tube 20 and one lens faces away from the interior 23. The optical element 30 may include a lens having two curved faces. For example, the optical element 30 may include a biconvex or spherical lens. In such an embodiment, the optical element 30 may include two curved faces, one of which faces away from the interior 23 of the tube 20, and one of which faces towards the interior 23 of the tube 20. In one embodiment, the optical element 30 may include a window facing the interior 23 of the tube 20 and a lens facing away from the interior 23. In such an embodiment, the optical element 30 may include a flat face facing towards the interior 23 of the tube 20 and a curved face facing away from the interior 23 of the tube 20. In one embodiment, the optical element 30 may include two windows, one of which faces away from the interior 23 of the tube 20, and one of which faces towards the interior 23 of the tube 20. In such an embodiment, the optical element 30 may include a flat face facing towards the interior 23 of the tube 20 and a flat face facing away from the interior 23 of the tube 20. In one embodiment, the optical element 30 may include multiple facets that may face towards or away from the interior of the tube 23. For example, the optical element 30 may include a prismatic-type shape.

The optical element 30 may focus electromagnetic radiation emitted by an area of interest towards the interior 23 of the tube 20. In one embodiment, the optical element 30 may focus electromagnetic radiation provided by a light source disposed near the second end 24 of the tube 20 away from the interior 23 of the tube 20. For example, as described in greater detail below, the optical element 30 may focus electromagnetic radiation provided by a light source disposed near the second end 24 of the tube 20 towards the area of interest. In various embodiments, the optical element 30 may focus electromagnetic radiation through the window 36. More generally, the optical element 30 may direct light from a first surface of the optical element 30, e.g. from a surface facing away from the interior 23 of the probe 10, to a second surface of the optical element 30, e.g. to a surface facing the interior of the probe 10.

A variety of materials may be used to construct the optical element 30. For example, the optical element 30 may be constructed from diamond, germanium, glass, plastic, potassium bromide, quartz, sapphire, silicon, sodium chloride, zinc selenide, zinc sulfide, other materials suitable for an optical element, and a combination of the foregoing. In one embodiment, the optical element 30 may be at least partially constructed from a salt known to those of ordinary skill in the art by the acronym KRS-5.

A variety of schemes may be used to construct the optical element 30. As shown in FIG. 1, the optical element 30, including the window 36 and the lens 38, may be integrally formed. More specifically, the window 36 and the lens 38 may be formed as a one-piece or unitary element. Alternately, the optical element 30 may include separately formed windows and lenses attached to each other using an adhesive, a fastener, or other conventional schemes. Potentially advantageously, by including an optical element 30 having an integrally formed window 36 and lens 38, the optical probe 10 may provide improved optical throughput compared to an optical probe including a separate window and lens.

The optical probes described herein may be used for a variety of applications. In one embodiment, the optical probes described herein may be used for attenuated total reflectance (ATR). In one embodiment, the optical probes described herein may be used for Raman spectroscopy and other types of spectroscopy for detecting electromagnetic radiation emitted by an area of interest.

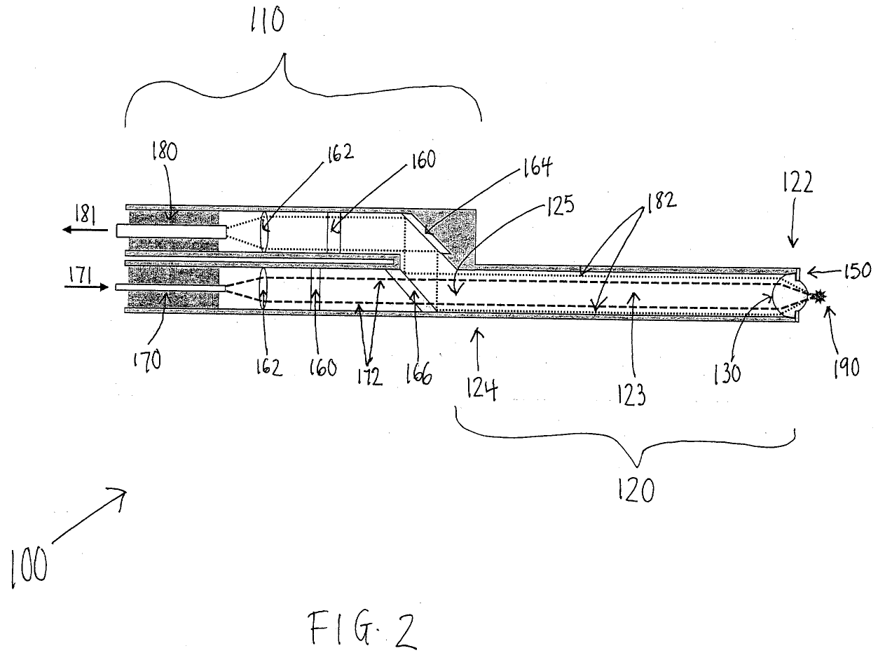

An exemplary system for Raman spectroscopy including an exemplary embodiment of an optical probe described herein is shown in FIG. 2. As shown in FIG. 2, the exemplary system 100 may include an optics housing 110, a tube 120, an optical element 130, a cap 150, and a spring between the optical element 130 and the cap 150. As shown in FIG. 2, the tube 120 may include a first end 122 for receiving the optical element 130, the spring, and the cap 150, and a second end 124 for interfacing with the optics housing 110. The tube 120, optical element 130, spring, and cap 150 may be constructed by using schemes similar to those described above.

The optics housing 110 and the tube 120 may be integrally formed. More specifically, the optics housing 110 and the tube 120 may be formed as a one-piece or unitary element. In one embodiment, the optics housing 110 and the tube 120 may be formed separately, and may be attached to each other using an adhesive, a fastener, or other conventional schemes.

As shown in FIG. 2, the optics housing 110 and the tube 120 may include a lumen 125 that permits transmission of electromagnetic radiation between the optics housing 110 and the tube 120. As further shown in FIG. 2, the optics housing 110 may include filters 160, lenses 162, a mirror 164, and a beam splitter 166 for transmitting electromagnetic radiation through the optics housing 110.

In the embodiment shown in FIG. 2, the optics housing 110 may include an excitation optical fiber 170 for providing electromagnetic radiation to an area of interest 190 through the optics housing 110 and the tube 120 and a collection optical fiber 180 for collecting electromagnetic radiation emitted by the area of interest 190.

Operation of the exemplary system 100 shown in FIG. 2 may be understood in the following manner. A light source, for example, a laser or other monochromatic source, may provide electromagnetic radiation to the excitation optical fiber 170 along a path denoted by arrow 171. The excitation optical fiber 170 may provide the electromagnetic radiation to the optics housing 110, and the electromagnetic radiation may pass through the tube 120 and the optical element 130 along a path denoted by long dashed-short dashed lines 172 to the area of interest 190. In response to being excited by the electromagnetic radiation provided by the exemplary system 100, the area of interest 190 may emit its own electromagnetic radiation. The characteristic electromagnetic radiation emitted by the area of interest 190 may be detected and focused by the optical element 130 to the interior 123 of the tube 120 and propagated through the optics housing 110 to the collection fiber 180 along a path denoted by dashed-dotted lines 182. The collection fiber 110 may then provide the characteristic electromagnetic radiation to a light detector, for example, a camera and/or a spectral analyzer, such as a spectrometer, along a path denoted by arrow 181. The spectrometer may include light selecting optics, such as a spectrograph or a filter, and a detector, such as a photodiode, a photomultiplier tube, or a charge-coupled device (CCD) camera.

As previously described, potentially advantageously, the optical probes described herein may provide substantially fluid tight seals between tubes and optical elements that may withstand a variety of hostile environments. For example, the optical probes described herein may be compatible with types of spectroscopy in which probes are placed directly into closed pathways, such as closed pathways found in chemical production feed lines, polymer extruders, reactors, and other industrial processes, to detect electromagnetic radiation. Chemical production feed lines, polymer extruders, reactors, and other industrial processes may be characterized by hostile environments that can include, for example, temperature cycling, pressure cycling, mechanical shock or vibration, and/or other external forces, such as corrosion and viscous fluid flow. Potentially advantageously, the schemes described herein for attaching the optical element 30 and the cap 50 to the tube 20 may provide optical probes that can inhibit the effects of hostile environments. Potentially advantageously, therefore, the optical probes described herein may withstand the hostile environments of feed lines and other types of hostile environments.

While the optical probes described herein have been particularly shown and described with reference to the exemplary embodiments thereof, those of ordinary skill in the art will understand that various changes may be made in the form and details herein without departing from the spirit and scope of the disclosure. Those of ordinary skill in the art will recognize or be able to ascertain many equivalents to the exemplary embodiments described specifically herein by using no more than routine experimentation. Such equivalents are intended to be encompassed by the scope of the present disclosure and the appended claims.

1. An optical probe comprising:
a tube having a first end, a second end, and an inner surface, at least a portion of the tube inner surface being tapered;
an optical element being insertable into the tube through the first end and having an outer surface, at least a portion of the optical element outer surface being tapered, the tapered portion of the optical element outer surface being mated to the tapered portion of the tube inner surface to provide a substantially fluid tight seal between the tube and the optical element;
a cap attachable to the first end; and,
a spring compressible between the optical element and the cap.

2. The optical probe of claim 1, wherein the second end includes an aperture permitting transmission of electromagnetic radiation out of the tube.

3. The optical probe of claim 1, wherein at least a portion of the tube inner surface is stepped, the spring being removeably and replaceably insertable into the stepped portion of the tube inner surface.

4. The optical probe of claim 3, wherein the stepped portion of the tube inner surface is disposxed adjacent to the tapered portion of the tube inner surface.

5. The optical probe of claim 3, wherein the stepped portion of the tube inner surface is stepped outward from the tube inner surface.

6. The optical probe of claim 1, wherein the tapered portion of the tube inner surface includes a taper extending outward from the tube inner surface.

7. The optical probe of claim 1, wherein the tapered portion of the tube inner surface includes a taper that spans a planar angle less than approximately 10 degrees.

8. The optical probe of claim 1, wherein the tapered portion of the tube inner surface includes a taper that spans a planar angle of approximately 3 degrees.

9. The optical probe of claim 1, wherein at least one of the tapered inner surface of the tube and the tapered outer surface of the optical element includes at least one of a gasket and a layer of at least one of gold, graphite, platinum, and polytetrafluoroethylene (PTFE).

10. The optical probe of claim 1, wherein the optical element includes at least one of a window for transmitting electromagnetic radiation and a lens for focusing electromagnetic radiation.

11. The optical probe of claim 1, wherein the spring comprises at least one of a spring washer, a C-ring, and a metal-loaded gasket.

12. The optical probe of claim 1, wherein the cap is attached to the first end using at least one of an adhesive, a braze, a fastener, a thread, and a weld.

13. The optical probe of claim 1, wherein the cap is welded to the first end under a load of at least approximately 150 psi.

14. The optical probe of claim 1, wherein the cap comprises a washer.

15. The optical probe of claim 1, wherein the tube comprises at least one of a ceramic, a polymer, and a metal.

16. The optical probe of claim 1, wherein the optical element comprises at least one of diamond, germanium, glass, plastic, potassium bromide, quartz, sapphire, silicon, sodium chloride, zinc selenide, and zinc sulfide.

17. An optical probe comprising:
a tube having a first end, a second end, and an inner surface, at least a portion of the tube inner surface being tapered; and,
an optical element being insertable into the tube through the first end and having an outer surface, at least a portion of the optical element outer surface being tapered, the tapered portion of the optical element outer surface being mated to the tapered portion of the tube inner surface to provide a substantially fluid tight seal between the tube and the optical element, the optical element including a window for transmitting electromagnetic radiation and a lens for focusing electromagnetic radiation.

18. The optical probe of claim 17, wherein the window and the lens are integrally formed.

19. The optical probe of claim 17, wherein the window and the lens are attached to each other using at least one of an adhesive and a fastener.

20. An optical probe comprising:
a tube having a first end, a second end, and an inner surface, at least a portion of the tube inner surface being tapered;
an optical element being insertable into the tube through the first end and having an outer surface, at least a portion of the optical element outer surface being tapered, the tapered portion of the optical element outer surface being mated to the tapered portion of the tube inner surface to provide a substantially fluid tight seal between the tube and the optical element, the optical element including a window for transmitting electromagnetic radiation and a lens for focusing electromagnetic radiation, the window and the lens being integrally formed;
a cap welded to the first end; and,
a spring washer compressible between the optical element and the cap.
